(19) United States
(12) Patent Application Publication
(10) Pub. No.: US 2011/0034992 A1
(43) Pub. Date: Feb. 10, 2011

(54) Stent and Method of Coating Same

(76) Inventor: John E. Papp, Temecula, CA (US)

Correspondence Address: SQUIRE, SANDERS & DEMPSEY LLP, 275 BATTERY STREET, SUITE 2600, SAN FRANCISCO, CA, 94111-3356, US

(21) Appl. No.: 12/535,556

(22) Filed: Aug. 4, 2009

(51) Int. Cl. A61F 2/06; B05D 3/00

(52) U.S. Cl. 623/1.16; 427/2.25

(57) ABSTRACT

Coating a stent may include continuously rotating the stent in one direction while spraying a first coating layer followed by continuously rotating the stent in another direction while spraying a second coating layer, wherein the first layer is preferentially distributed over a side surface of the stent struts and the second layer is preferentially distributed over an opposite side surface of the stent struts. The overall coating distribution combining both layers may be evenly distributed over the two side surfaces of the stent struts.

### FIELD OF THE INVENTION

Briefly and in general terms, the present invention generally relates to coating a medical device, more specifically, to a stent and method for forming a desired coating distribution.

### BACKGROUND OF THE INVENTION

In percutaneous transluminal coronary angioplasty (PTCA), a balloon catheter is inserted through a brachial or femoral artery, positioned across a coronary artery occlusion, and inflated to compress against atherosclerotic plaque to open, by remodeling, the lumen of the coronary artery. The balloon is then deflated and withdrawn. Problems with PTCA include formation of intimal flaps or torn arterial linings, both of which can create another occlusion in the lumen of the coronary artery. Moreover, thrombosis and restenosis may occur several months after the procedure and create a need for additional angioplasty or a surgical bypass operation. Stents are used to address these issues. Stents are small, intricate, implantable medical devices and are generally left implanted within the patient to reduce occlusions, inhibit thrombosis and restenosis, and maintain patency within vascular lumens such as, for example, the lumen of a coronary artery.

The treatment of a diseased site or lesion with a stent involves both delivery and deployment of the stent. Stent delivery refers to introducing and transporting the stent through an anatomical lumen to a desired treatment site, such as a lesion in a vessel. An anatomical lumen can be any cavity, duct, or a tubular organ such as a blood vessel, urinary tract, and bile duct. Stent deployment corresponds to expansion of the stent within the anatomical lumen at the region requiring treatment. Delivery and deployment of a stent are accomplished by positioning the stent about one end of a catheter, inserting the end of the catheter through the skin into an anatomical lumen, advancing the catheter in the anatomical lumen to a desired treatment location, expanding the stent at the treatment location, and removing the catheter from the lumen with the stent remaining at the treatment location.

In the case of a balloon expandable stent, the stent is mounted about a balloon disposed on the catheter. Mounting the stent typically involves compressing or crimping the stent onto the balloon prior to insertion in an anatomical lumen. At the treatment site within the lumen, the stent is expanded by inflating the balloon. The balloon may then be deflated and the catheter withdrawn from the stent and the lumen, leaving the stent at the treatment site. In the case of a self-expanding stent, the stent may be secured to the catheter via a retractable sheath. When the stent is at the treatment site, the sheath may be withdrawn which allows the stent to self-expand.

Stents are often modified to provide drug delivery capabilities to further address thrombosis and restenosis. Stents may be coated with a polymeric carrier impregnated with a drug or therapeutic substance. A conventional method of coating includes applying a composition including a solvent, a polymer dissolved in the solvent, and a therapeutic substance dispersed in the blend to the stent by immersing the stent in the composition or by spraying the composition onto the stent. The solvent is allowed to evaporate, leaving on the stent strut surfaces a coating of the polymer and the therapeutic substance impregnated in the polymer.

The application of a uniform coating with good adhesion to a substrate can be difficult for small and intricate medical devices, such as certain stents for coronary and peripheral arteries. Such stents can be quite small. Stents for the coronary vessel anatomy typically have an overall diameter of only a few millimeters and a total length of several millimeters. Stents for the peripheral vessel anatomy are generally greater in diameter and length. Such peripheral stents may have a diameter up to 10 mm and a length of up to 200 mm. These stents may be constructed of a fine mesh network of struts, which provide support or push against the walls of the anatomical lumen in which the stent is implanted.

For example, FIG. 11 shows an upper portion of a stent 10 having an overall body shape that is hollow and tubular. The stent can be made from wires, fibers, coiled sheet, with or without gaps, or a scaffolding network of rings. The stent can have any particular geometrical configuration, such as a sinusoidal or serpentine strut configuration, and should not be limited to what is illustrated in FIG. 11. The variation in stent patterns is virtually unlimited. The stent can be balloon expandable or self-expandable, both of which are well known in the art.

FIGS. 11 and 12 show stents with two different stent patterns. The stents are illustrated in an uncrimped or expanded state. In both FIGS. 11 and 12, the stent 10 includes many interconnecting struts 12, 14 separated from each other by gaps 16. The struts 12, 14 can be made of any suitable material, such as a biocompatible metal or polymer. The polymer may also be bioabsorbable. The stent 10 has an overall longitudinal length 40 measured from opposite ends, referred to as the distal and proximal ends 22, 24. The stent 10 has an overall body 50 having a tube shape with a central passageway 17 passing through the entire longitudinal length of the stent. The central passageway has two circular openings, there being one circular opening at each of the distal and proximal ends 22, 24 of the overall tubular body 50. A central axis 18 runs through the central passageway in the center of the tubular body 50. At least some of the struts 12 are arranged in series to form sinusoidal or serpentine ring structures 20 that encircle the central axis 18.

FIG. 13 is an exemplary cross-sectional view of the stent 10 along line 13-13 in FIG. 12. There can be any number of struts 12, 14 along line 13-13, which runs perpendicular to the central axis 18 of the stent 10. In FIG. 13, the cross-section of seven struts 12, 14 are shown for ease of illustration. The struts 12, 14 in cross-section are arranged in a circular pattern having an outer diameter 26 and an inner diameter 28. The circular pattern encircles the central axis 18. A portion of the surface of each strut faces radially inward in a direction 30 facing toward the central axis 18. A portion of the surface of each strut faces radially outward in a direction 32 facing away from the central axis 18. The various strut surfaces that face radially outward collectively form the outer surface 34 of the stent 10. The various strut surfaces that face radially inward collectively form the inner surface 36 of the stent 10.

The terms “axial” and “longitudinal” are used interchangeably and relate to a direction, line or orientation that is parallel or substantially parallel to the central axis of a stent or a central axis of a cylindrical structure. The term “circumferential” relates to the direction along a circumference of a stent or a circular structure. The terms “radial” and “radially” relate to a direction, line or orientation that is perpendicular or substantially perpendicular to the central axis of a stent or a central axis of a cylindrical structure.

Coating of the thin network of struts often leads to non-uniform coating thickness. In many stent applications, it is desired to have a coating thickness that is uniform or evenly distributed over the various surfaces of the stent struts. A uniform coating thickness helps ensure that the drug is released evenly in the region of the anatomical lumen being treated.

There is a continuing need for a system and a method for coating medical devices that are efficient and reliable.

### SUMMARY OF THE INVENTION

Briefly and in general terms, the present invention is directed to a system and method for coating a medical device. In some aspects of the present invention, a method for coating a medical device involves rotating the medical device in a rotational direction while applying a first coating layer followed by rotating the medical device in an opposite rotational direction while applying a second coating layer in order to form a desired coating distribution over various surfaces of the medical device.

In aspects of the present invention, a method for coating a stent comprises discharging from a dispenser a first coating substance onto the stent while simultaneously rotating the stent around a longitudinal axis of the stent in a first rotation direction and while simultaneously moving a dispenser across a longitudinal length of the stent. The method further comprises, discharging from the dispenser a second coating substance onto the first coating substance on the stent while simultaneously rotating the stent around the longitudinal axis of the stent in a second rotation direction and while simultaneously moving the dispenser across the longitudinal length of the stent. In other aspects of the present invention, the method further comprises drying the first coating substance discharged onto the stent while simultaneously rotating the stent, the drying of the first coating substance is performed as an intervening step between the discharging of the first coating substance and discharging of the second coating substance. In further aspects of the present invention, the rotating of the stent, simultaneously with drying the first coating substance, is in the first rotation direction, and the rotating of the stent, simultaneously with drying of the second coating substance, is in the second rotation direction.

In aspects of the present invention, a method for coating a stent comprises performing at least two process cycles, each process cycle including spraying a coating substance onto or into a stent while simultaneously rotating the stent, the rotating of the stent during at least one of the process cycles is in a rotation direction that is opposite of a rotation direction of at least one other of the process cycles. In further aspects, each process cycle is a spray-dry cycle in which the spraying is followed by drying the coating substance on the stent, the drying includes rotating the stent while blowing a gas onto the coating substance on the stent. In detailed aspects, the rotating of the stent includes rotating the stent around a longitudinal axis of the stent, the longitudinal axis extending from a proximal end of the stent to a distal end of the stent.

In aspects of the present invention, a method for coating a stent comprises performing at least two process cycles, each process cycle including distributing a sprayed coating substance onto or into a stent while simultaneously rotating the stent. Performing the at least two process cycles includes balancing the distribution of the coating substance on or within a plurality of struts of the stent, by rotating the stent during at least one of the process cycles in a rotation direction that is opposite of a rotation direction of at least one other of the process cycles.

In aspects of the present invention, an implantable medical device comprises a plurality of struts arranged in a circular pattern, each of the struts includes a first side surface facing in a first circumferential direction, a second side surface facing in a second circumferential direction opposite the first circumferential direction, and a coating over the first and second side surfaces, the coating for each strut having a plurality of layers including a first layer and a second layer over and around the first layer, the first layer having an average thickness over the first side surface that is greater than that on the second side surface, the second layer having an average thickness over the second side surface that is greater than that over the first side surface.

The features and advantages of the invention will be more readily understood from the following detailed description which should be read in conjunction with the accompanying drawings.

### BRIEF DESCRIPTION OF THE DRAWINGS

FIG. 1 is a diagram of a system for coating a medical device, showing a medical device carrier in a spray area located adjacent a drying area.

FIG. 2 is a diagram of the system of FIG. 1, showing the medical device carrier in the drying area.

FIGS. 3A-3C are radial cross-sectional views, FIGS. 3A and 3C showing a stent strut covered by a coating, and FIG. 3B showing a plurality of stent struts arranged around the stent central axis.

FIGS. 4A-4D are diagrams of a system for coating a medical device, showing a sequence of steps in a spray-dry cycle for forming a first coating layer on the medical device.

FIGS. 5A-5D are diagrams of the system of FIGS. 4A-4D, showing a sequence of steps in a subsequent spray-dry cycle for forming a second coating layer over the first coating layer.

FIG. 6 is a radial cross-sectional view of a stent strut, showing a coating that is distributed substantially evenly over opposite circumferential side surfaces of the strut.

FIG. 7A is a radial cross-sectional view of a stent strut, showing a first coating layer distributed more heavily over one side of strut, and a second coating layer distributed more heavily over the opposite side of the strut.

FIG. 7B is a radial cross-sectional view of the strut of FIG. 7A, showing the overall coating distributed substantially evenly over opposite circumferential side surfaces of the strut.

FIG. 8 is a plot of averages of thickness measurements taken over several points around a stent strut.

FIGS. 9 and 10 are photographs of radial cross-sections of stent struts showing the location of measurements for FIG. 8

FIG. 11 is a perspective view of a portion of a stent.

FIGS. 12 and 13 are perspective and cross-sectional views, respectively, of a stent.

### DETAILED DESCRIPTION OF THE PREFERRED EMBODIMENTS

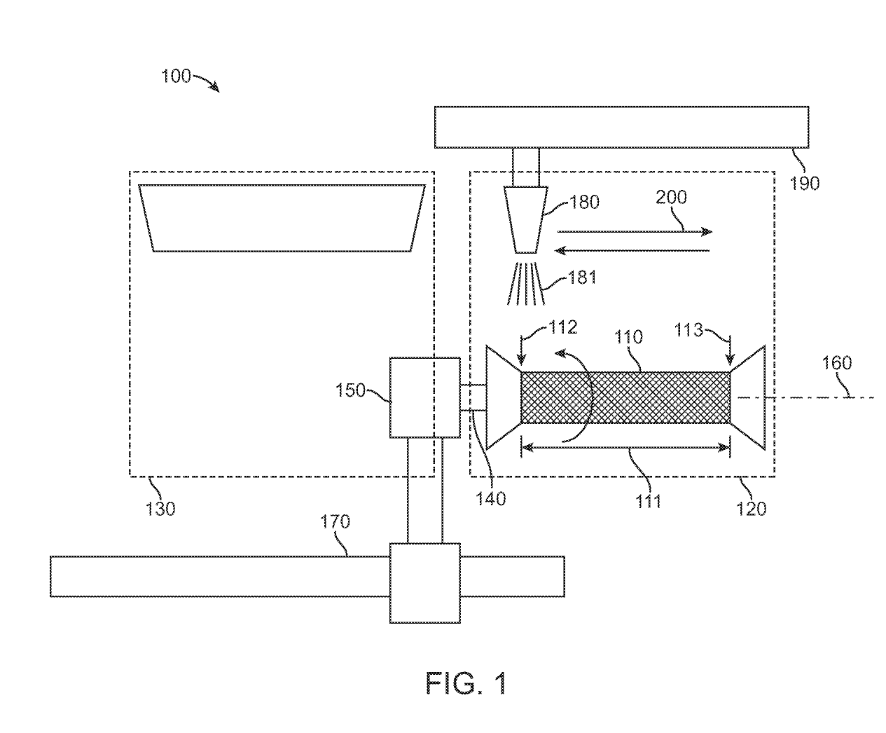

Referring now in more detail to the exemplary drawings for purposes of illustrating embodiments of the invention, wherein like reference numerals designate corresponding or like elements among the several views, there is shown in FIG. 1 a stent coating system 100 in which a stent 110 is moved back and forth between a spraying area 120 and a drying area 130.

The stent 110 is sprayed with a coating substance in the spraying area 120, then moved to the drying area 130 where the stent is dried at least partially with a heated gas. The stent is rotated continuously about its central axis during the spraying and drying steps. Rotation helps to ensure that all surfaces of the stent are brought into the flow path of the coating substance and the heated gas, thereby enhancing uniformity of distribution of the coating substance on the stent.

The process of spraying followed by drying is referred to as one “spray-dry” cycle. The spray-dry cycle is repeated any number of times until the stent carries a desired thickness of coating. The drying step removes some of the solvents in the coating layer previously applied to the stent, which makes the coating layer a more stable substrate onto which the next coating layer may be deposited.

Referring again to FIG. 1, the stent 110 is mounted horizontally on a carrier 140 rotatably engaged to a motor 150 which rotates the carrier and the stent about the central axis 160 of the stent while the stent is simultaneously being coated and while the stent is subsequently dried. The carrier 140 is slideably engaged to a first guide assembly 170 that moves the carrier and the stent in and out of the spraying and drying areas 120, 130.

A coating dispenser 180 is disposed within the spraying area 120. The coating dispenser 180 is slideably engaged to a second guide assembly 190. The second guide assembly 190 moves the coating dispenser 180 horizontally across the entire longitudinal length 111 of the stent 110, starting from the proximal end 112 of the stent to the distal end 113 of the stent, while the coating dispenser 180 simultaneously discharges a coating substance 181 downward onto the stent and while the motor 150 simultaneously rotates the stent. The coating dispenser may move along a path that is longer than the longitudinal length 111 so that movement of the dispenser “overshoots” or extends beyond the opposite ends of the stent, thereby eliminating end effects from a cone shaped spray plume. The coating dispenser 180 is moved by the second guide assembly 190 in a horizontal direction 200 that is parallel or substantially parallel to the central axis 160 of the stent to help ensure that the proximal and distal portions of the stent receive the same amount of coating.

When the coating dispenser 180 reaches or passes the distal end 113 of the stent 110, the coating dispenser 180 reverses direction and moves back toward the proximal end 112. During this time, the stent 110 continues to rotate in the same direction. The coating substance 181 is discharged as small droplets distributed in a conical spray plume that gradually thins with increasing distance from the coating dispenser 180. As such, spray conditions differ according to distance from the coating dispenser 180. Thus, continuous rotation of the stent helps to ensure that all surfaces of the stent are subjected to the same spray conditions.

The process of moving the coating dispenser 180 from the proximal end 112 to the distal end 113 and back to the proximal end is referred to as a “two-pass” spray process since the coating dispenser discharges the coating substance across the length 111 of the stent 110 twice.

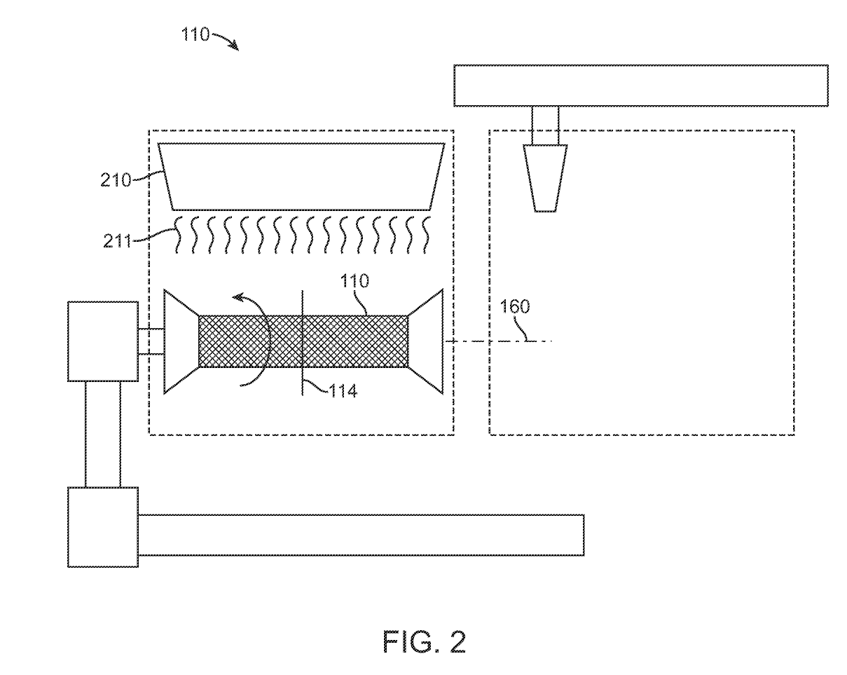

As shown in FIG. 2, after the two-pass spray process is completed, the stent 110 is moved from the spraying area 120 to the drying area 130, where the gas dispenser 210 discharges a gas 211 onto the stent. The stent 110 is rotated continuously while the gas is discharged onto it. The stent 110 is rotated in the same direction as in the spraying area 120.

It will be appreciated that the amount of drying and evaporation that occurs depends in part on velocity and temperature of the gas that travels over the wet coating layer on the stent, and that there is a velocity gradient and a temperature gradient in the gas flow path with increasing distance from the gas dispenser 210. As such, drying conditions differ according to distance from the gas dispenser 210. Thus, continuous rotation of the stent helps ensure that all surfaces of the stent are subjected to the same drying conditions.

Modifications can be made to the process described above in connection with FIGS. 1 and 2. For example more than two spray passes can be completed by the coating dispenser for each spray step prior to proceeding to a drying step.

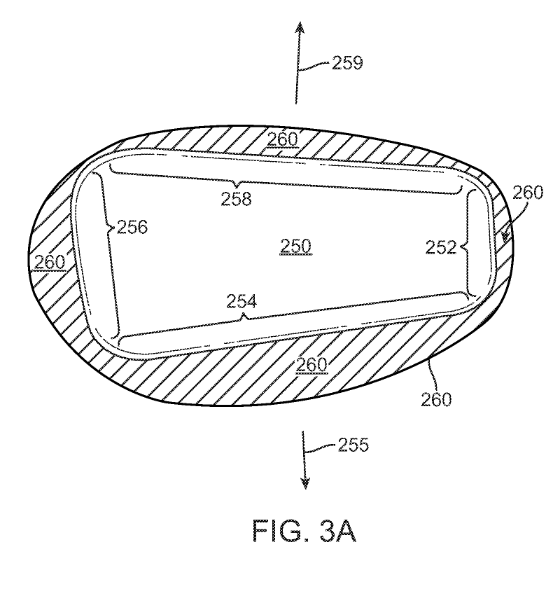

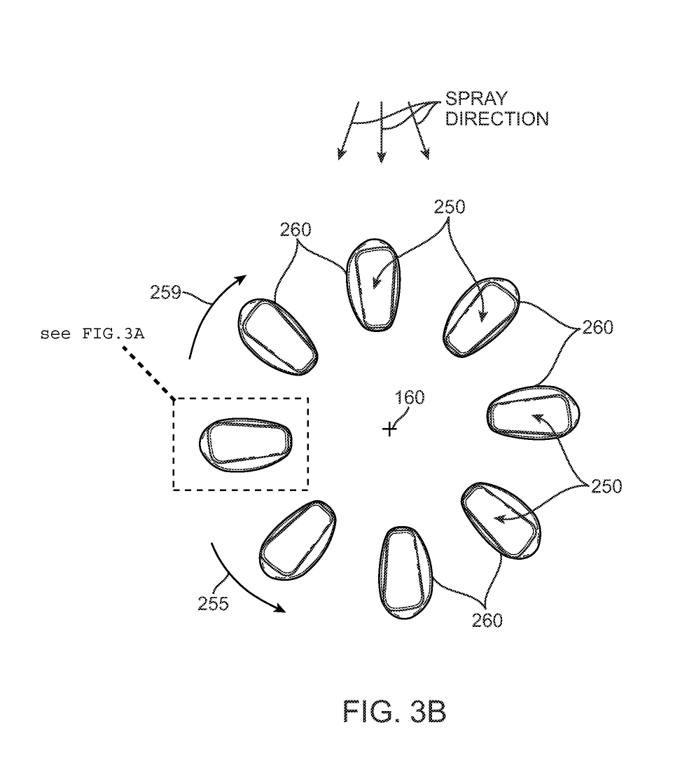

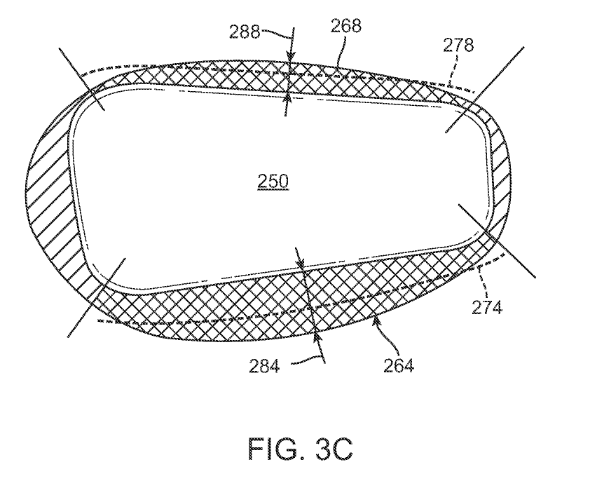

FIGS. 3A-3C show an exemplary cross-section of stent struts in a cut plane perpendicular to the stent longitudinal axis 160 after multiple spray-dry cycles are performed as described above in connection with FIGS. 1 and 2. In FIGS. 3A and 3C, the radially outward facing surface of the strut (corresponding to the stent outer diameter) is on the left side of the illustrated cross-section, and the radially inward facing surface of the strut (corresponding to the stent inner diameter) is on the right side of the illustrated cross-section.

As shown in FIG. 3A, the above describe spray-dry cycle with the two-pass process in which the stent is rotated continuously in the one direction helps to ensure that all surfaces of the stent strut 250 are covered with a coating 260, thereby enhancing uniformity of distribution of the coating substance on the stent 110. The coating 260 is illustrated with hatch lines. The coating 260 is the result of one or more spray-dry cycles, wherein the stent 110 is rotated in the same rotational direction for all spray-dry cycles. When only one spray-dry cycle is performed, the coating 260 consists of only one coating layer. When multiple spray-dry cycles are performed, the coating 260 is the accumulation of all coating layers, each individual layer formed with an individual spray-dry cycle. Typically, the number of layers ranges from three to seventy, though any number of layers are within the scope of the present invention.

Without being limited to a particular theory of operation, it is believed that the distribution of the coating substance around individual stent struts depends on a variety of processing parameters. Processing parameters including without limitation the rate of rotation of the stent relative to the velocity of spray droplets, rate of linear movement of the coating dispenser across the length of the stent, distance of the coating dispenser from the stent, spray angle relative to the stent central axis (e.g., perpendicular or at another angle), spray alignment relative to the stent central axis (e.g., centered or offset to one side), spray plume direction (e.g., vertical upward, vertical downward, or horizontal), size of the spray plume relative to the stent diameter, and other spray plume characteristics. Spray plume characteristics include without limitation the degree of atomization of the coating substance in a spray plume, the distribution of coating droplets in the spray plume, and shape of the spray plume.

Additional processing parameters that may affect the balance of coating distribution around stent struts include without limitation temperature and humidity of air surrounding the stent or of any gas blown onto the stent during spraying and drying, air turbulence or direction of laminar air flow around the stent as it is being sprayed, the composition of constituents within the coating substance, and the physical characteristics of the constituents. The composition of constituents includes without limitation the relative proportions of solvent, polymer carrier, and drug in the coating substance. Relevant physical characteristics of the constituents include without limitation viscosity, solubility, and vapor pressure as it relates to rate of evaporation.

FIG. 3A shows a radial cross-section of the stent strut 250 on a cut plane 114 (FIG. 2) that is substantially perpendicular to the central axis 160 of the stent. The cross-section view is in a direction substantially parallel to the central axis 160 of the stent. The strut cross-section shown in FIG. 3A is representative of the multiple strut cross-sections shown in FIG. 3B arranged in a circular pattern around the central axis of the stent 160.

In FIG. 3B, eight stent struts are shown, though a stent generally may have any number of stent struts in a particular cut plane that is perpendicular to the stent central axis.

The stent strut 250 has a generally radially inward facing surface 252, which faces toward the central axis of the stent. A generally radially outward facing surface 256 faces away from the central axis of the stent. A first side surface 254 faces in a first circumferential direction 255. A second side surface 258 faces in a second circumferential direction 259 that is the opposite of the first circumferential direction 255.

As shown in FIG. 3A, the coating 260 may have a greater distribution of the coating substance over the first side surface 254 than over the second side surface 258 under certain combinations of processing parameters. Applicant has found that, in combination with other processing parameters, such an unbalanced distribution may occur with rotation of the stent in a single rotational direction during all spray-dry cycles.

The coating 260 in FIG. 3A is redrawn in FIG. 3C. In FIG. 3C, the coating 260 is illustrated with single- and cross-hatch lines to show adjoining segments of the coating. The coating 260 has a first thickness profile 264 over the first side surface 254 and a second thickness profile 268 over the second side surface 258. The thickness profiles 264, 268 are illustrated with double cross-hatching for clarity of illustration. The first thickness profile 264 is substantially greater than the second thickness profile 268.

The term “over,” as used in relation to the coating, refers to the portion of the coating located normal (i.e., perpendicular) to a strut surface. The term “thickness profile” refers to the area between a strut surface and a surface of the coating over the strut surface (or a covered surface of an individual layer within the coating). The term “mean thickness profile” refers to the average of two or more thickness profiles. The term “thickness,” when used alone in relation to the coating, refers to a distance measured from a strut surface to a surface of the coating over the strut surface (or a covered surface of an individual layer within the coating), wherein the distance is measured in a direction normal to the strut surface. The term “average thickness” refers to the average of thicknesses over a strut surface, unless specified otherwise.

Still referring to FIG. 3C, the coating 260 has a first average thickness 274 over the first side surface 254, and a second average thickness 278 over the second side surface 258. The average thicknesses 274, 278 are shown as dashed-lines over the strut surface. The first average thickness 274 is substantially greater than the second average thickness 278. The coating 260 has a first maximum thickness 284 over the first side surface 254, and a second maximum thickness 288 over the second side surface 258. The first maximum thickness 284 is substantially greater than the second maximum thickness 288.

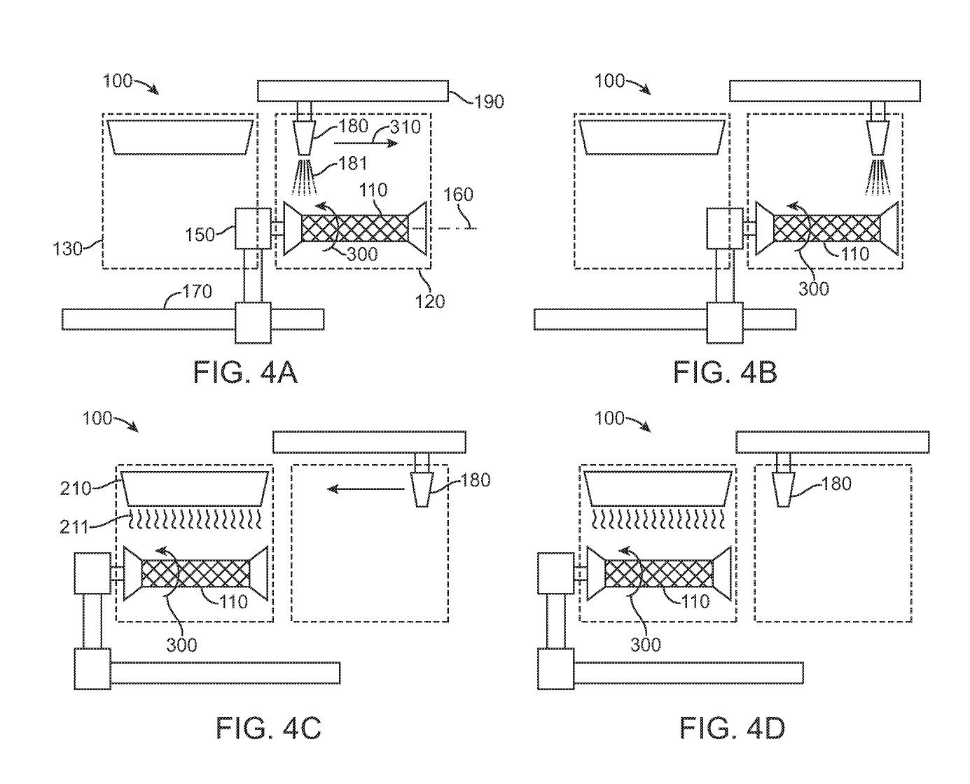

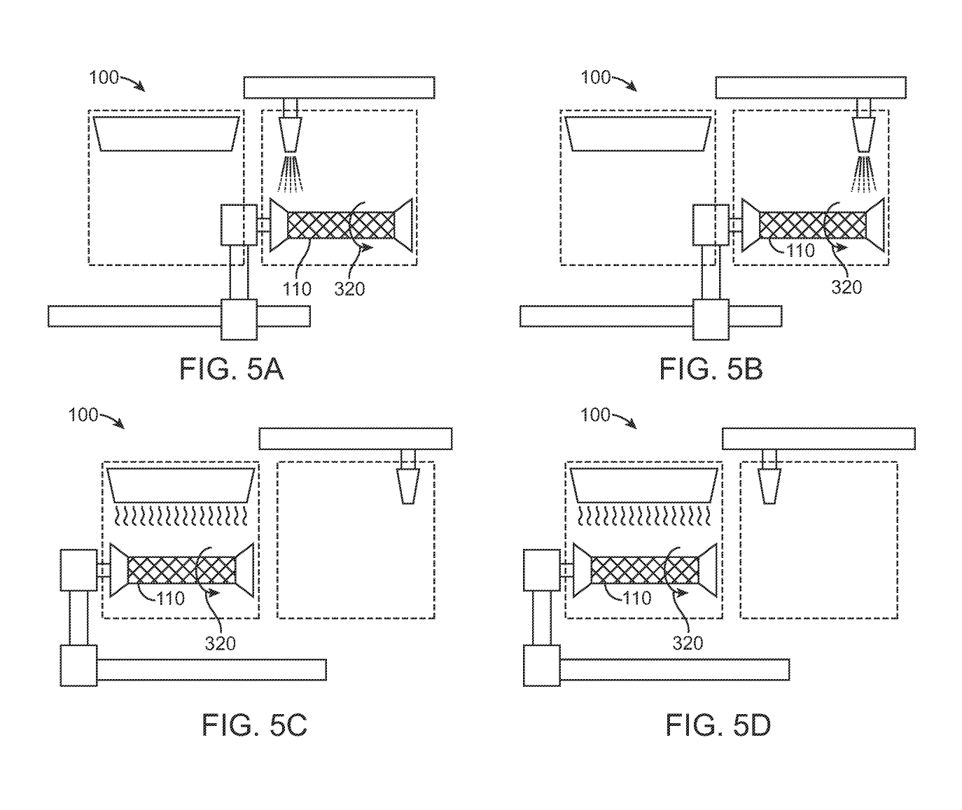

With a combination of processing parameters, distribution of the coating around the stent struts may be balanced between the first side surface 254 and the second side surface 258. Applicant has unexpected found that, in combination with other processing parameters, balancing between the first and second side surfaces 254, 258 may be performed by alternating the rotational direction of the stent between spray-dry cycles. For example, a first spray-dry cycle may be performed with the stent rotated continuously in a first rotational direction, as shown in FIGS. 4A-4D, then a next spray-dry cycle may be performed with the stent rotated continuously in a second rotational direction opposite to the first rotational direction, as shown in FIGS. 5A-5D.

In FIGS. 4A-4D and 5A-5D, the coating dispenser 180 is oriented to project droplets of the coating substance in a conical spray plume. The spray plume is projected in a vertical, downward direction, wherein the spray plume is substantially centered over the diameter of the stent. The central axis of the conical spray plume is substantially perpendicular to the stent central axis 160, and the stent central axis is substantially horizontal. The central axis of the conical spray plume intersects the stent central axis 160 so as to be aligned with the stent central axis, as opposed to being offset to one side of the stent central axis.

Referring again to FIG. 4A, the stent 110 is in the spraying area 120 where the coating dispenser 180 is discharging the coating substance 181 onto the stent 110 while the stent is simultaneously rotating in a first rotational direction 300 around the central axis 160 of the stent. While discharging the coating substance and rotating the stent in the first rotational direction, the coating dispenser 180 is moved from a first end segment of the stent, as shown in FIG. 4A, to a second end segment of the stent, as shown in FIG. 4B. As a result, the coating substance 181 is distributed over and around the struts of the stent. In some embodiments, the coating dispenser 180 is moved along a direction 310 that is parallel or substantially parallel to the central axis 160 of the stent 110.

The coating dispenser 180 starts its linear movement while spraying at a location that is to the left of the end of the stent. At this start position, the leading edge of the spray plume is not on the stent, which allows the spray plume to stabilize before it contacts the stent. The coating dispenser 180 finishes its linear movement at a finish position. At the finish position, the trailing edge of the spray plume has moved beyond the opposite end of the stent. The starting and finish positions define a travel path that exceeds the longitudinal length of the stent, thereby allowing the end segments of the stent to receive as much coating substance as the middle segment of the stent and thereby enhancing coating distribution uniformity.

FIGS. 4C and 4D show the stent 110 in the drying area 130, and shows the gas dispenser 210 blowing gas 211 onto the stent while the stent continues to rotate in the first rotational direction 300. The gas 211 dries the first coating layer on the stent. In FIG. 4D, the coating dispenser 180 has returned to the same position it occupied in FIG. 4A.

FIG. 5A-5D shows a repeat of the steps of FIG. 4A-4D except the stent 110 is rotated continuously in a second rotational direction 320, which is opposite the first rotational direction 300. For example, the first rotational direction may be clockwise and the second rotational direction may be counterclockwise. In FIGS. 5A and 5B, the coating substance is sprayed onto the first coating layer to form a second coating layer over and around the first coating layer.

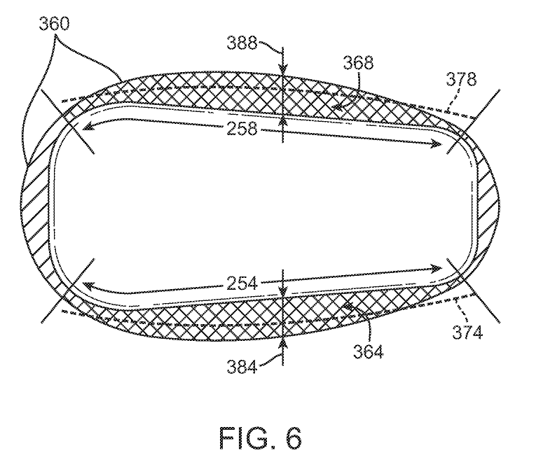

FIG. 6 shows a cross-section of a stent strut in a cut plane perpendicular to the stent central axis 160 after multiple spray-dry cycles, each cycle performed as described above in connection with FIGS. 4A-4D and 5A-5D, with the rotational direction of the stent being reversed after each spray-dry cycle. It is to be understood that any number of spray-dry cycles may be performed, with the rotational direction of the stent being reversed after each spray-dry cycle, until a desired amount of coating substance is carried by the stent 110.

As shown in FIG. 6, with some combinations of processing parameters, the coating 360 may have a substantially balanced distribution of the coating substance over the first side surface 254 and the second side surface 258. The phrase “substantially balanced distribution” refers to similarity of size, or shape, or both size and shape of the individual thickness profiles over the first side surface 254 and the second side surface 258. In FIG. 6, the thickness profiles are similar in size and shape. Size may be characterized by cross-sectional area.

The coating 360 has a first thickness profile 364 over the first side surface 254 and a second thickness profile 368 over the second side surface 258. The thickness profiles 364, 368 over the circumferential side surfaces are illustrated with double cross-hatching for clarity of illustration. The first thickness profile 364 is substantially the same as the second thickness profile 368. The coating 360 has a first average thickness 374 over the first side surface 254, and a second average thickness 378 over the second side surface 258. The average thicknesses 374, 378 are shown as dashed-lines over the strut surface. The first average thickness 374 is substantially the same as the second average thickness 378. The coating has a first maximum thickness 384 over the first side surface 254, and a second maximum thickness 388 over the second side surface 258. The first maximum thickness 384 is substantially the same as the second maximum thickness 388.

It will be appreciated that modifications could be made to the above described methods. In the illustrated embodiment of FIGS. 4A-4D and 5A-5D, the coating dispenser 180 makes one spray pass across the longitudinal length of the stent, whereby it moves only in one direction (left to right) when coating the stent. The coating dispenser does not return to its starting position shown in FIG. 4A while the coating substance 181 is sprayed onto the stent.

In other embodiments, the coating dispenser performs multiple spray passes over the stent during the spray process of a spray-dry cycle. After each spray pass, the directional rotation of the stent is reversed. When the stent moves to the drying area, the stent will have multiple coating layers having been applied with alternating stent rotational directions, and the multiple coating layers will be dried together, as opposed to being dried individually as in a case where an intervening drying step is performed between each spray pass.

In other embodiments, the coating dispenser moves in two linear directions while spraying the stent. For example, as the stent is rotated in the spray area, the coating dispenser makes one left-to-right spray pass across the longitudinal length of the stent, then the coating dispenser remains in place within the spraying area. For the next spray-dry cycle, as the stent is rotated in the opposite direction in the spray area, the coating dispenser makes one right-to-left spray pass across the longitudinal length of the stent.

In other embodiments, the coating dispenser returns to its starting position while the coating substance 181 is sprayed onto the stent. Thus, the coating dispenser makes two spray passes across the longitudinal length of the stent (left to right, then right to left) while the stent rotates in the same direction.

In some embodiments, the rotational direction of the stent is reversed after multiple spray-dry cycles during which the stent is rotated in only one rotational direction. For example, multiple spray-dry cycles can be performed with the stent rotating continuously in the first rotational direction 300, followed by multiple spray-dry cycles with the stent rotating continuously in the second rotational direction 320. The number of spray-dry cycles for each rotational direction can be selected to balance the distribution of the coating substance over the circumferential side surfaces.

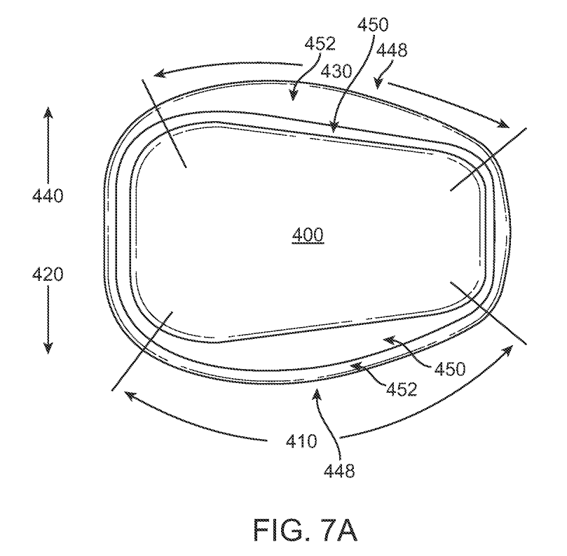

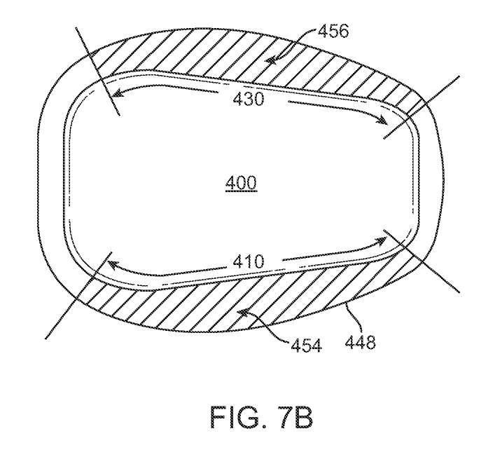

In some embodiments, a plurality of stent struts are arranged in a circular pattern around the stent central axis. As shown in FIG. 7A, each stent strut 400 includes a first side surface 410 facing in a first circumferential direction 420, a second side surface 430 facing in a second circumferential direction 440 opposite the first circumferential direction. Each of the struts also includes a coating 448 over the first and second side surfaces.

The coating has a first layer 450 and a second layer 452 over and around the first layer. The first layer 450 may be formed from one or more spray-dry cycles in which the stent is rotated in a first rotational direction. The second layer 452 may be formed from one or more spray-dry cycles in which the stent is rotated in a second rotational direction. The first layer 450 has an average thickness over the first side surface that is substantially greater than that on the second side surface. The second layer 452 has an average thickness over the second side surface that is substantially greater than that over the first side surface.

The coating in FIG. 7A is redrawn in FIG. 7B. The coating 448 (first and second layers combined) has a first thickness profile 454 and a second thickness profile 456. The first and second thickness profiles are illustrated with hatch lines for clarity of illustration. The first thickness profile 454 includes the cross-sectional area of all the layers over the first side surface 410. The second thickness profile 456 includes the cross-sectional area of all the layers over the second side surface 430. The first thickness profile 448 is same or substantially the same in area and shape as the second thickness profile 448.

It is to be understood the coating distribution described in connection with FIGS. 7A and 7B could be created in various ways. For example, the first layer 450 could be formed by spraying the coating substance at an acute angle relative to the stent central axis, the angle selected to preferentially coat the first side surface 410, and the second layer 452 could be formed by spraying the coating substance at a second acute angle relative to the stent central axis, the second angle selected to preferentially coat the second side surface 430. As a further example, the first layer 450 could be formed by orienting the stent vertically with the first side surface 410 facing vertically upward while the spray coating is discharged horizontally toward the stent, and the second layer 452 could be formed by orienting the stent vertically with the second side surface 430 facing vertically upward while the spray coating is discharge horizontally toward the stent. In a further non-limiting example, the first layer 450 could be formed by a spray-dry cycle during which the stent is coated in only one direction, and the second layer 452 could be formed by a subsequent spray-dry cycle during which the stent is rotated in the opposite direction.

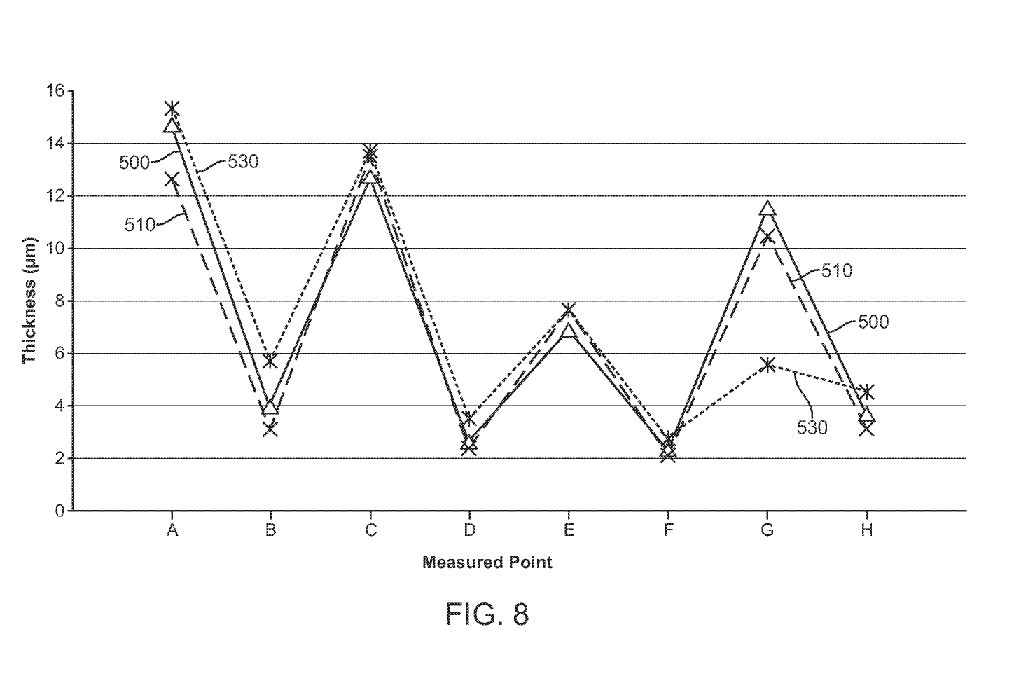

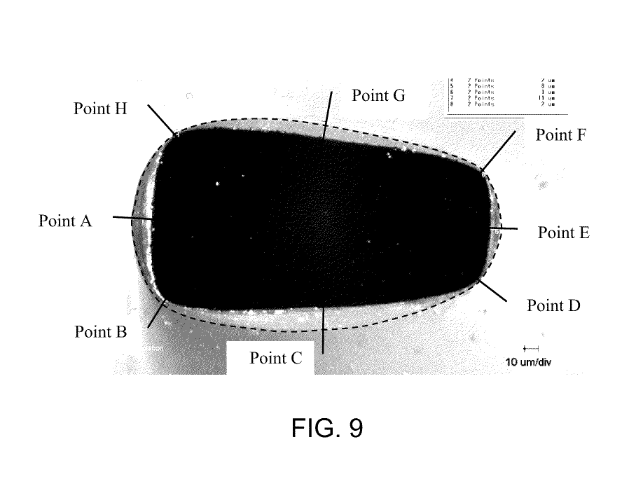

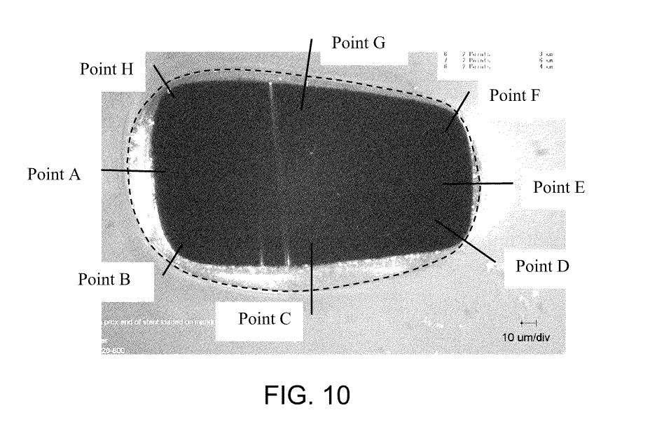

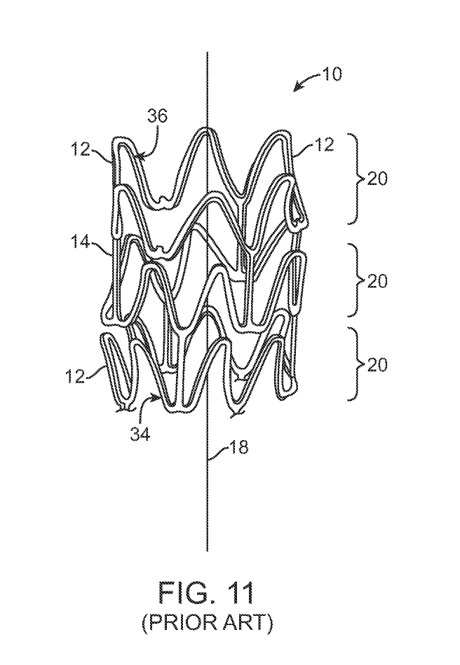

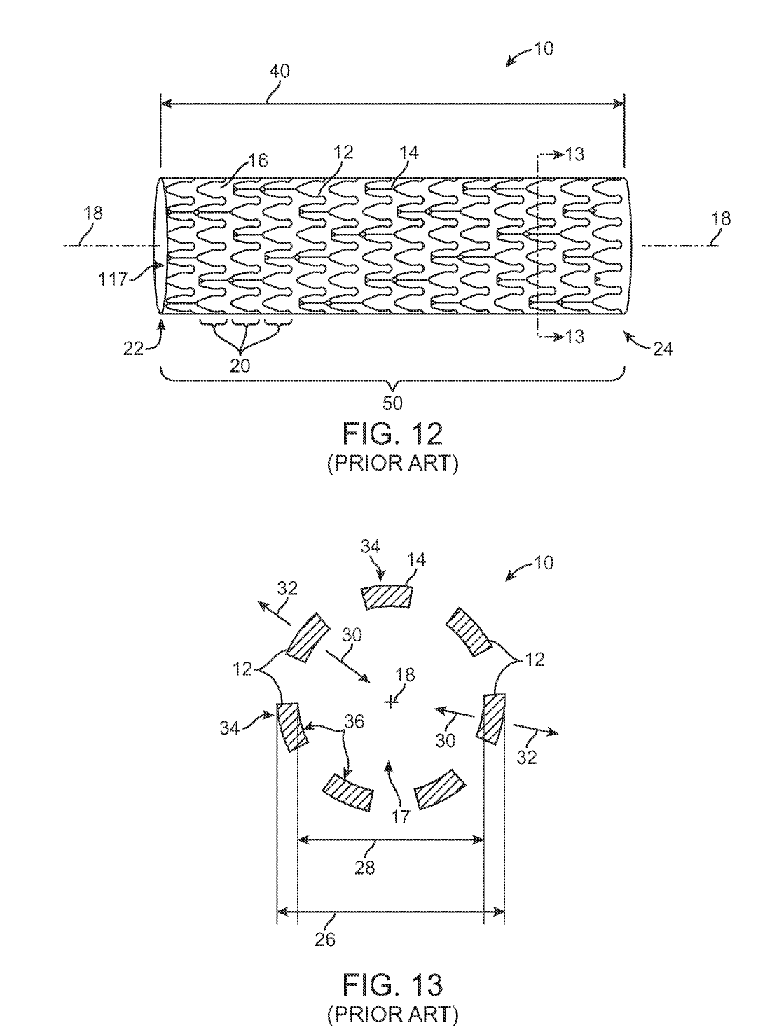

FIG. 8 shows a plot of thickness measurements taken along various points (A, B, C, D, E, F, G and H) around a stent strut. Data points for averages of multiple thickness measurements are shown in the vertical axis versus measurement location on the horizontal axis. As shown in FIGS. 9 and 10, point A corresponds to the approximate center of the radially outward facing surface. Point B corresponds to the approximate boundary between the radially outward facing surface and a first side surface. Point C corresponds to the approximate center of the first side surface. Point D corresponds to the approximate boundary between the first side surface and the radially inward facing surface. Point E corresponds to the approximate center of the radially inward facing surface. Point F corresponds to the approximate boundary between the radially inward facing surface and a second side surface facing in the opposite direction of the first side surface. Point G corresponds to the approximate center of the second side surface. Point H corresponds to the approximate boundary between the second side surface and the radially outward facing surface.

For the data shown in FIG. 8, the spray plume was projected in a vertical, downward direction and the spray plume was substantially centered over the diameter of the stent. The central axis of the conical spray plume was substantially perpendicular to the horizontal stent central axis.

Lines 500 and 510 in FIG. 8 represent “reverse rotation” cases and show thickness measurements around stent struts subjected to multiple spray-dry cycles in which the stent was continuously rotated during spraying. The rotation direction was reversed after each spray-dry cycle. For the spray step in each spray-dry cycle, the stent was rotated continuously while a spray nozzle performed one spray pass across the entire stent longitudinal length. Line 500 represents averages of thickness measurements taken after twenty-two spray-dry cycles forming a coating including a drug followed by fourteen spray-dry cycles forming a final coating including no drug. Line 510 represents averages of thickness measurements taken after fifteen spray-dry cycles forming a coating including a drug followed by ten spray-dry cycles forming a final coating including no drug.

FIG. 9 shows a photograph of a radial cross-section of one of the stent struts for which measurement data was included in line 5 10. In FIG. 9, the stent strut appears as the dark center and the outer surface of the coating around the stent strut is outlined with a dashed line.

Line 530 in FIG. 8 represents a “single rotational direction” case and shows averages for thickness measurements around stent struts subjected to multiple spray-dry cycles in which the stent was continuously rotated in the same direction for all spray-dry cycles. The direction of rotation was not reversed for any of the spray-dry cycles. During spraying, the stent was rotated continuously while a spray nozzle performed two spray passes: a first spray pass in one linear direction followed by a second spray pass in the opposite linear direction across the entire stent longitudinal length. Line 530 represents averages of thickness measurements taken after twenty-two spray-dry cycles forming a coating including a drug followed by fourteen spray-dry cycles forming a final coating including no drug.

FIG. 10 shows a photograph of a radial cross-section of one of the stent struts for which measurement data was included in line 530. In FIG. 10, the stent strut appears as the dark center and the outer surface of the coating around the stent strut is outlined with a dashed line.

In the single rotation case of line 530, as indicated by FIGS. 8 and 10, the thickness average over Point C on the first circumferential side surface of the stent strut is substantially greater than the thickness average over Point G on the second circumferential side surface. During spraying, the first circumferential side surface rotated toward the spray nozzle while it faced the nozzle, and the second circumferential side surface rotated away from the spray nozzle while it faced the nozzle. That is, the first circumferential side surface was always moving toward the spray droplets when it was being coated, and the second circumferential side surface was always moving away from the spray droplets when it was being coated.

In the reverse rotation cases of lines 500 and 510, as indicated by FIGS. 8 and 9, the thickness average over Point C on the first circumferential side surface is substantially equal to the thickness average over Point G on the second circumferential side surface. For half of the spray-dry cycles, the first circumferential side surface rotated toward the spray nozzle while it was being coated, and the second circumferential side surface rotated away from the spray nozzle while it was being coated. For the other half of the spray-dry cycles, the first circumferential side surface rotated away from the spray nozzle while it was being coated, and the second circumferential side surface rotated toward the spray nozzle while it was being coated.

Without being limited to a particular theory of operation, it is believed that as rotation rate of the stent approaches the velocity of the spray coating droplets, surfaces moving away from the spray nozzle will tend to receive a lesser amount of coating substance, thereby creating an imbalance in coating distribution between opposite circumferential side surfaces. Although decreasing the rate of stent rotation may increase uniformity in the coating distribution. Applicant has found that decreasing the rate of stent rotation is accompanied by an increase in the amount of spray coating substance that accumulates on radially outward facing surfaces of the strut, creating a coating distribution imbalance between radially outward and inward facing surfaces. Also, decreasing the rate of stent rotation may also cause the coating substance to pool and web at regions of the stent framework where spacing between stent struts is relatively small, such as where adjoining stent struts meet at acute angles.

In some embodiments of the invention, the rotation rate of the stent about its central axis is selected, at least in part, so as to reduce the incidence of coating substance pooling in between stent struts, and spray-dry cycles with rotation reversals between cycles may be performed to allow for a balanced distribution of the stent coating between circumferential side surfaces.

In FIGS. 9 and 10, the stent strut is made of a substantially non-porous material. The above described processes may be performed on stents made of porous materials and materials that absorb liquids. Such stents may include struts formed of metallic or polymeric powder that have been sintered together under heat and/or pressure in such a manner that voids, cavities, and/or pores are distributed on the surface or entirely through the strut cross-section. The distribution of the coating substance inside the stent can be controlled in accordance with the processes described herein. For example, the depth to which a coating penetrates into a first surface of the strut, as a result of stent rotation, can be balanced or made equal to the depth to which the coating penetrates into another surface of the strut, by rotating the stent in an opposite rotational direction.

The penetration profile of the coating can be controlled as desired for struts formed of a porous material. The “penetration profile” is the area between the strut structural surface and the coating penetration boundary below the strut surface, the boundary being the interface between internal regions of the strut having no coating and internal regions of the strut in which the coating is present. The penetration profile below a circumferential side surface can be purposely made larger than the penetration profile below an opposite circumferential side surface by rotating the stent only in one rotational direction during spraying. Also, the rotation profiles on opposite circumferential surfaces made to be substantially equal to each other in shape, or size, or both shape and size, by alternating the rotational direction of the stent while it is being sprayed with the coating.

In some embodiments, an implantable medical device comprises a plurality of porous struts arranged in a circular pattern, each of the struts includes a first side surface facing in a first circumferential direction, and a second side surface facing in a second circumferential direction opposite the first circumferential direction. The strut includes regions having no therapeutic substance. The strut also includes a therapeutic substance within regions of the strut beneath the first and second side surfaces. The therapeutic substance has a penetration profile under the first side surface that is substantially the same in area or shape as that under the second side surface.

In some embodiments of the invention, the rotation rate of the stent about its central axis is selected, at least in part, so as to reduce the disparity in coating distribution between radially outward and inward facing surfaces, and spray-dry cycles with rotation reversals between cycles may be performed to reduce disparity in coating distribution between circumferential side surfaces. In some embodiments, the therapeutic substance is contained fully, or essentially fully, within the stent strut. In some embodiments, the therapeutic substance is disposed partially within the stent strut, so that it is partially below the stent strut surface and partially above the stent strut surface.

In some embodiments, the coating that is sprayed onto the strut contains a drug and solvent, but does not contain a polymer carrier for the drug. In this case, the build up of therapeutic drug is balanced, with regard to shape and/or cross-sectional area, over both side walls of the stent struts.

In some embodiments, there is no intervening drying step between the spray passes. The stent is sprayed multiple times with the rotational direction being reversed after one or more spray passes. This may, for example, be performed for a coating composition with constituents having a high vapor pressure (or low boiling point) which allows the coating to dry relatively quickly. For example, a coating containing a solvent, in which a drug and/or polymer are dissolved to facilitate spraying, may evaporate at a sufficiently high rate to allow multiple spray passes to be performed without any intervening drying step, so that the effect on thickness profiles and coating distributions that are obtained are substantially the same as those shown in FIGS. 3A, 3C, 6, 7A, and 7B. Drying occurs during the spraying step as opposed to during a dedicated, intervening drying step. The term “intervening drying step” refers to a period of time where spraying is discontinued to facilitate or induce drying, and may or may not include blowing a gas onto the coating.

While several particular forms of the invention have been illustrated and described, it will also be apparent that various modifications can be made without departing from the scope of the invention. For example, a modification can be made to one or more of the processing parameters described above, including without limitation the spray angle relative to the stent central axis (e.g., perpendicular or at another angle), spray alignment relative to the stent central axis (e.g., centered or offset to one side), spray plume direction (e.g., vertical upward, vertical downward, or horizontal), size of the spray plume relative to the stent diameter, and other spray plume characteristics. It is also contemplated that various combinations or subcombinations of the specific features and aspects of the disclosed embodiments can be combined with or substituted for one another in order to form varying modes of the invention. Accordingly, it is not intended that the invention be limited, except as by the appended claims.

What is claimed is:

1. A method of coating a stent, the method comprising:
discharging from a dispenser a first coating substance onto the stent while simultaneously rotating the stent around a longitudinal axis of the stent in a first rotation direction and while simultaneously moving a dispenser across a longitudinal length of the stent; followed by
discharging from the dispenser a second coating substance onto the first coating substance on the stent while simultaneously rotating the stent around the longitudinal axis of the stent in a second rotation direction and while simultaneously moving the dispenser across the longitudinal length of the stent, the second rotation direction being the reverse of the first rotation direction.

2. The method of claim 1, wherein the first and second coating substances have the same composition of constituents.

3. The method of claim 1, wherein the discharging of the first coating substance includes discharging droplets of the first coating substance from the dispenser.

4. The method of claim 1, further comprising:
drying the first coating substance discharged onto the stent while simultaneously rotating the stent, the drying of the first coating substance is performed as an intervening step between the discharging of the first coating substance and discharging of the second coating substance.

5. The method of claim 4, wherein the rotating of the stent, simultaneously with drying the first coating substance, is in the first rotation direction.

6. The method of claim 4, wherein the drying of the first coating substance includes discharging a first gas onto the stent simultaneously with rotating the stent in the first direction.

7. The method of claim 1, wherein the moving of the dispenser across the longitudinal length of the stent is in a direction that is parallel or substantially parallel to the longitudinal axis of the stent.

8. The method of claim 1, wherein the stent includes a tubular framework of struts, the tubular framework having a central passageway that extends from a proximal end of the tubular framework to a distal end of the tubular framework, and the longitudinal axis of the stent extends from the proximal end to the distal end and through the central passageway.

9. A method of coating a stent, the method comprising:
performing at least two process cycles, each process cycle including spraying a coating substance onto or into a stent while simultaneously rotating the stent, the rotating of the stent during at least one of the process cycles is in a rotation direction that is opposite of a rotation direction of at least one other of the process cycles.

10. The method of claim 9, wherein each process cycle is a spray-dry cycle in which the spraying is followed by drying the coating substance on the stent, the drying includes rotating the stent while blowing a gas onto the coating substance on the stent.

11. The method of claim 9, wherein the rotating of the stent includes rotating the stent around a longitudinal axis of the stent, the longitudinal axis extending from a proximal end of the stent to a distal end of the stent.

12. The method of claim 9, wherein the spraying of the coating substance onto the stent, while simultaneously rotating the stent, includes spraying the coating substance out of a dispenser while simultaneously moving the dispenser across the longitudinal length of the stent.

13. The method of claim 9, wherein the spraying of the coating substance onto the stent includes spraying the coating substance in a downward direction that is substantially perpendicular to the longitudinal axis of the stent.

14. The method of claim 9, wherein the stent includes a plurality of struts arranged in a circular pattern, each strut includes a first side surface facing in a first circumferential direction and a second side surface facing in a second circumferential direction opposite the first circumferential direction, the spraying of the coating substance during one of the process cycles includes forming around each of the struts a coating layer having a greater distribution of the coating substance over the first side surfaces than on the second side surfaces as a result of rotation of the stent in a first direction, and the spraying of the coating substance during another one of the process cycles includes forming around each of the struts a coating layer having a greater distribution of the coating substance over the second side surfaces than on the first side surfaces as a result of rotation of the stent in a second direction that is the reverse of the first direction.

15. A method of coating a stent, the method comprising:
performing at least two process cycles, each process cycle including distributing a sprayed coating substance onto or into a stent while simultaneously rotating the stent,
wherein performing the at least two process cycles includes balancing the distribution of the coating substance on or within a plurality of struts of the stent, by rotating the stent during at least one of the process cycles in a rotation direction that is opposite of a rotation direction of at least one other of the process cycles.

16. The method of claim 15, wherein each of the plurality of struts includes a first side surface facing in a first circumferential direction and a second side surface facing in a second circumferential direction opposite the first circumferential direction, and the balancing of the distribution of the coating substance on the plurality of struts includes forming a coating around each of the struts, the coating having a mean thickness profile over the first side surfaces that is the same or substantially the same as a mean thickness profile over the second side surfaces.

17. The method of claim 15, wherein each of the plurality of struts includes a first side surface facing in a first circumferential direction and a second side surface facing in a second circumferential direction opposite the first circumferential direction, and the balancing of the distribution of the coating substance on the plurality of struts includes:
forming a first coating layer around the struts during one or more of the process cycles, the first coating having an average thickness over the first side surfaces that is substantially greater than that on the second side surfaces; and
forming a second coating layer around the first coating during another one or more of the process cycles, the second coating having an average thickness over the second side surfaces that is substantially greater than that on the first side surfaces.

18. The method of claim 15, wherein, for each process cycle, the distributing of the coating substance is followed by drying the coating substance on the stent, and the drying includes rotating the stent while blowing a gas onto the coating substance on the stent.

19. The method of claim 15, wherein, for each process cycle, the rotating of the stent includes rotating the stent around a rotational axis extending from a proximal end of the stent to a distal end of the stent.

20. The method of claim 15, wherein, for each process cycle, the distributing of the coating substance onto the stent includes spraying the coating substance from a dispenser while moving the dispenser from a proximal end of the stent to a distal end of the stent.

21. The method of claim 15, wherein, for each process cycle, the distributing of the coating substance onto the stent includes spraying the coating substance in a downward direction that is substantially perpendicular to the axis of rotation of the stent.

22. An implantable medical device comprising:
a plurality of struts arranged in a circular pattern, each of the struts includes a first side surface facing in a first circumferential direction, a second side surface facing in a second circumferential direction opposite the first circumferential direction, and a coating over the first and second side surfaces, the coating for each strut having a plurality of layers including a first layer and a second layer over and around the first layer, the first layer having an average thickness over the first side surface that is greater than that on the second side surface, the second layer having an average thickness over the second side surface that is greater than that over the first side surface.

23. The medical device of claim 22, wherein the coating for each strut has a first thickness profile that includes all the layers over the first side surface and a second thickness profile that includes all the layers over the second side surface, the first thickness profile being the same or substantially the same in cross-sectional area as the second thickness profile.

24. The medical device of claim 22, wherein the plurality of stent struts forms a tubular framework having a proximal end, a distal end, and a central passageway extending from the proximal end to the distal end, the plurality of stent struts arranged in the circular pattern around an axis extending from the proximal end to the distal end.

25. The medical device of claim 22, wherein the coating for each strut is partially disposed within the strut, the coating having a first penetration profile below the first side surface and a second penetration profile below the second side surface, the first penetration profile being the same or substantially the same in cross-sectional area as the second penetration profile.
